(19) United States
(12) Patent Application Publication
(10) Pub. No.: US 2025/0082239 A1
(43) Pub. Date: Mar. 13, 2025

(54) SYSTEMS AND METHODS OF MANAGING SUBSTANCE USE DISORDER OF A PERSON BASED ON DETECTED NEUROPHYSIOLOGICAL ACTIVITY DATA

(71) Applicant: UNIVERSITY OF MARYLAND, BALTIMORE COUNTY, BALTIMORE, MD (US)

(72) Inventors: Ramana Vinjamuri, Pine Brook, NJ (US); Abhijit Dutt, Cockeysville, MD (US); Nilanjan Banerjee, Elkridge, MD (US)

(21) Appl. No.: 18/748,153

(22) Filed: Jun. 20, 2024

Related U.S. Application Data
(60) Provisional application No. 63/510,163, filed on Jun. 26, 2023.

(51) Int. Cl. A61B 5/16; A61B 5/384

(52) U.S. Cl. CPC A61B 5/16; A61B 5/384

(57) ABSTRACT

Systems and methods of managing substance use disorder of a person based on detected neurophysiological activity data are disclosed. According to an aspect, a system includes a biosensor configured to acquire neurophysiological activity data of a person. The system also includes a computing device comprising a substance use disorder manager configured to implement an application that interfaces with the person for managing the person's substance use disorder. The manager is also configured to receive the acquired neurophysiological activity data. Further, the manager is configured to determine that the person reached a substance abuse trigger point based on the acquired neurophysiological activity data. The manager is also configured to operate a user interface to present an interactive function of the application for distracting the person in response to determining that the person reached the subject abuse trigger point.

### CROSS REFERENCE TO RELATED APPLICATION

This application claims priority to U.S. Provisional Patent Application No. 63/510,163, filed Jun. 26, 2023, and titled INTEGRATED SYSTEM FOR REAL-TIME DETECTION AND MANAGEMENT OF SUBSTANCE USE DISORDER, the content of which is incorporated herein by reference in its entirety.

### TECHNICAL FIELD

The presently disclosed subject matter relates generally to healthcare and medical systems. Particularly, the presently disclosed subject matter relates to systems and methods of managing substance use disorder of a person based on detected neurophysiological activity data.

### BACKGROUND

Substance use disorder (SUD) is a dangerous disease that affects an individual's brain and behavior. This leads to uncontrolled use of illicit drugs, alcohol, excessive use of legal drugs or other addictive behaviors. The prevalence and the rate of increase of SUD in the United States deem it a rapidly growing epidemic. Furthermore, during COVID19 pandemic, one of the serious challenges faced by the American Society of Addiction Medicine (ASAM) is the treatment of homeless individuals with SUD because of their compromised immune systems. The National Institute on Drug Abuse (NIDA) estimates that the total expenditure of drug-related complications exceeds 500 billion dollars when healthcare costs and job losses are considered. Despite this growing epidemic and its subsequent consequences, there are limited management and treatment options, pharmacotherapies, and psychosocial treatments available for SUD. To this end NIDA's mission and strategic plan emphasize the importance of the development of new and improved strategies and treatments for detection and management of SUD.

In view of the foregoing, there is a continuing need for improved systems and techniques for assisting individuals with managing their SUD.

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1 is a block diagram of a system for managing SUD of a person in accordance with embodiments of the present disclosure;

FIG. 2 is a block diagram of a system for managing SUD of a person in accordance with embodiments of the present disclosure;

FIG. 3 is a flow diagram of a method for managing SUD of a person in accordance with embodiments of the present disclosure;

FIG. 4 is a flow diagram of another method for managing SUD of a person in accordance with embodiments of the present disclosure;

FIG. 5 is a diagram showing RespiBAN Professional's placement of electrodes;

FIG. 6 are depictions of two protocols tested under a study;

FIG. 7 depicts a table showing the findings from an analysis;

FIGS. 8A and 8B depict a table showing classification accuracy using logistic regression with top three features;

FIG. 9 is a pie chart showing significance of the features based on how frequently they were used in three different classifiers for best accuracies;

FIGS. 10A and 10B depict a table showing accuracies and AUC for all types of classifications for all subjects using three algorithms and all features;

FIGS. 11A and 11B depict a table showing accuracies and AUC for all types of classifications for all subjects using three algorithms and the top three features;

FIG. 12 is a graph showing significance of the features based on how frequently they were used in three different classifiers for best accuracies;

FIG. 13 is a diagram of an example MIST paradigm;

FIG. 14 depicts data collection in (A), and EEG electrode location in (B);

FIG. 15 depicts a table showing data of features extracted from peripheral physiological signals;

FIG. 16 depicts a table of features extracted from EEG signals;

FIG. 17 depicts a table showing a comparison of classification accuracy across various approaches for discriminating non-stress, social stress, and mental stress using different classifiers;

FIG. 18 depicts a table showing the performance of multi-level stress detection using different classifiers;

FIG. 19 depicts images showing an initial understanding of the spatial distribution of essential features extracted from EEG across the brain;

FIG. 20 depicts images showing tomography of relative power activation from different EEG rhythms from subject 2, 60s timeframe; and

FIG. 21 shows graphs with feature importance of peripheral physiological features for multi-class and multi-level stress detection across different time durations.

### SUMMARY

The presently disclosed subject matter relates to systems and methods of managing substance use disorder of a person based on detected neurophysiological activity data. According to an aspect, a system includes a biosensor configured to acquire neurophysiological activity data of a person. The system also includes a computing device comprising a substance use disorder manager configured to implement an application that interfaces with the person for managing the person's substance use disorder. The manager is also configured to receive the acquired neurophysiological activity data. Further, the manager is configured to determine that the person reached a substance abuse trigger point based on the acquired neurophysiological activity data. The manager is also configured to operate a user interface to present an interactive function of the application for distracting the person in response to determining that the person reached the subject abuse trigger point.

### DETAILED DESCRIPTION

The following detailed description is made with reference to the figures. Exemplary embodiments are described to illustrate the disclosure, not to limit its scope, which is defined by the claims. Those of ordinary skill in the art will recognize a number of equivalent variations in the description that follows.

Articles “a” and “an” are used herein to refer to one or to more than one (i.e. at least one) of the grammatical object of the article. By way of example, “an element” means at least one element and can include more than one element.

“About” is used to provide flexibility to a numerical endpoint by providing that a given value may be “slightly above” or “slightly below” the endpoint without affecting the desired result.

The use herein of the terms “including,” “comprising,” or “having,” and variations thereof is meant to encompass the elements listed thereafter and equivalents thereof as well as additional elements. Embodiments recited as “including,” “comprising,” or “having” certain elements are also contemplated as “consisting essentially of” and “consisting” of those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a range is stated as between 1%-50%, it is intended that values such as between 2%-40%, 10%-30%, or 1%-3%, etc. are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

As used herein, the term “biosensor” refers to a sensor (or device) operable to detect and measure one or more indicators of a person's physiological or biological changes or conditions. Example biosensors include, but are not limited to, electroencephalography (EEG) sensors, electrodermal activity (EDA) sensors, electrodermal activity (EDA) sensors, body temperature sensors, or the like Ecological Momentary Assessment Data (EMA) sensors that can be acquired from smartphones. This data could include accelerometers, text data, text speed, location, temperature, proximity to water bodies or other geographical locations. Such biosensors can detect and measure physiological and biological changes in a person's body. An EDA sensor, for example, can measure the electrical conductance of the skin, which varies with its moisture level and is influenced by stress and arousal. A body temperature sensor, for example, can measure the body temperature of the person. An EEG sensor, for example, can measure the electrical activity of the brain. A biosensor may be configured to output an electrical signal indicative of the neurophysiological activity data and to communicate the electrical signal to the computing device.

As referred to herein, the term “neurophysiological activity data” refers to data that can be acquired from a person and can be used to assess the functioning and/or activity of the person's nervous system. Example measurements that can be acquired from the person, such as through suitable sensors, include electrical activity data, magnetic field data, blood flow and oxygenation, chemical signals, and autonomic responses. Electrical activity data can be data from electrical signals generated by neurons. Magnetic field data can be received from magnetic fields produced by neural activity. Blood flow and oxygenation data can indicate changes in blood flow and oxygen levels in the brain. Chemical signal data can indicate concentrations of various neurotransmitters or other chemicals in the nervous system. Autonomic response data relates to the autonomic nervous system, such as heart rate variability, skin conductance, and other physiological responses controlled by the autonomic nervous system. These and other data can be acquired by biosensors.

As referred to herein, the terms “computing device” and “entities” should be broadly construed and should be understood to be interchangeable. They may include any type of computing device, for example, a server, a desktop computer, a laptop computer, a smart phone, a cell phone, a pager, a personal digital assistant (PDA, e.g., with GPRS NIC), a mobile computer with a smartphone client, or the like.

As referred to herein, a user interface is generally a system by which users interact with a computing device. A user interface can include an input for allowing users to manipulate a computing device, and can include an output for allowing the system to present information and/or data, indicate the effects of the user's manipulation, etc. An example of a user interface on a computing device (e.g., a mobile device) includes a graphical user interface (GUI) that allows users to interact with programs in more ways than typing. A GUI typically can offer display objects, and visual indicators, as opposed to text-based interfaces, typed command labels or text navigation to represent information and actions available to a user. For example, an interface can be a display window or display object, which is selectable by a user of a mobile device for interaction. A user interface can include an input for allowing users to manipulate a computing device, and can include an output for allowing the computing device to present information and/or data, indicate the effects of the user's manipulation, etc. An example of a user interface on a computing device includes a graphical user interface (GUI) that allows users to interact with programs or applications in more ways than typing. A GUI typically can offer display objects, and visual indicators, as opposed to text-based interfaces, typed command labels or text navigation to represent information and actions available to a user. For example, a user interface can be a display window or display object, which is selectable by a user of a computing device for interaction. The display object can be displayed on a display screen of a computing device and can be selected by and interacted with by a user using the user interface. In an example, the display of the computing device can be a touch screen, which can display the display icon. The user can depress the area of the display screen where the display icon is displayed for selecting the display icon. In another example, the user can use any other suitable user interface of a computing device, such as a keypad, to select the display icon or display object.

As referred to herein, the term “substance use disorder” is a medical condition characterized by a person's inability to control the use of a substance despite harmful consequences. Such substances can include, but are not limited to, alcohol, illicit drugs, and prescription medications.

As referred to herein, the terms “application”, “app”, or “computer application” are used interchangeably and refer to a programmed set of instructions that can reside in memory of a computing device and designed to perform a specific set of tasks or functions for the user of the computing device. Some applications can operate with a user interface for allowing the user to interact with the application via windows, text boxes, menus, buttons, and the like. Also, some applications can play video, display images, play music or other sound, or the like. Applications can perform specific functions or tasks that can be idle until enabled for functioning by its application or instruction from the user or another application.

Disclosed herein are systems and methods for managing substance use disorder (SUD) of a person. A system in accordance with embodiments can be “all-in-one” and can provide real-time detection and management of SUD. In an example, a system can include a headband that has a combination of EEG and EDA biosensors (or electrodes) that measure the neurophysiological activity of a person or individual. The handband can transmit the measured activities to an application that runs on a smartphone of the person. The application can process the neurophysiological activity data for detecting a trigger point that may lead to substance abuse using artificial intelligence (AI) and/or machine learning (ML). Once a trigger point is detected, the application on the smartphone can provide distractors, such as videogames or other engagement that stimulate biofeedback and thus help in the management of SUD.

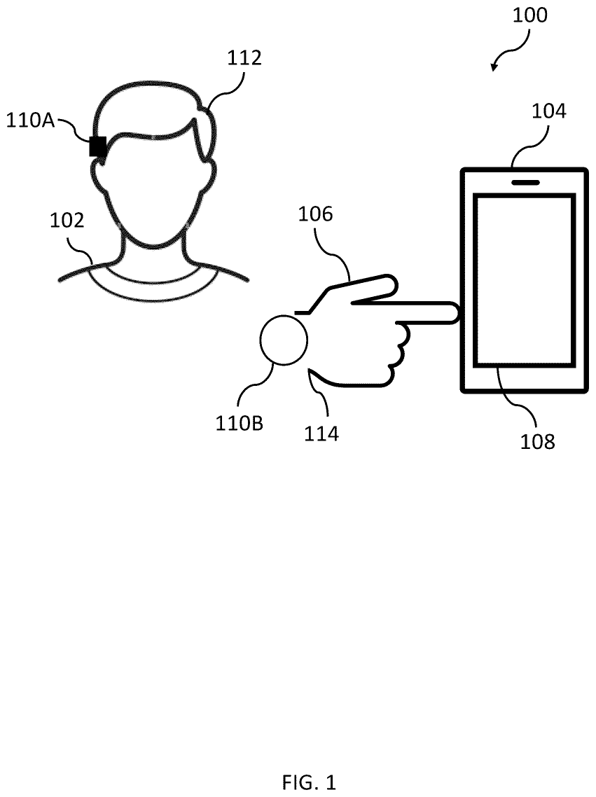

FIG. 1 illustrates a block diagram of a system, generally designated 100, for managing SUD of a person in accordance with embodiments of the present disclosure. Referring to FIG. 1, the system 100 is shown in an operational environment of use by a user 102. The system 100 can include a computing device 104 accessible by the user 102. The user 102 may interact with the computing device 104 via any suitable technique, such as, by use of the person's hand 106 to touch a touchscreen display 108 of the computing device 104. In this example, the computing device 104 is a smartphone but may alternatively be any other suitable computing device, such as a tablet computer or notebook computer. The computing device 104 can be configured to display graphics and text, or otherwise present information or graphics to the user 102. In addition, the computing device 104 can be configured to generate sounds (e.g., music or voice) via its speakers.

The system 100 also include one or more biosensors 110A, 110B. Example biosensors 110A, 110B include, but are not limited to, EEG sensors, EDA sensors (or electrodes), and the like. The biosensors 110 can be suitably worn by the person 102 such as on the head 112 (e.g., against the person's scalp) or wrist 114 of the person 102. Biosensor 110A can be worn on the person's head 112 via a strap or other attachment mechanism. Biosensor 110B can be strapped onto the person's wrist 114, or be part of a smartwatch worn by the person 112. Biosensors 110A, 110B can acquire neurophysiological activity data of the person 112 and output an electrical signal indicative of the neurophysiological activity data. Biosensors 110A, 110B or devices operatively connected thereto can communicate the signals to the computing device 104 via wired or wireless communication technologies (e.g., BLUETOOTH® communications technology) for SUD management in accordance with embodiments of the present disclosure.

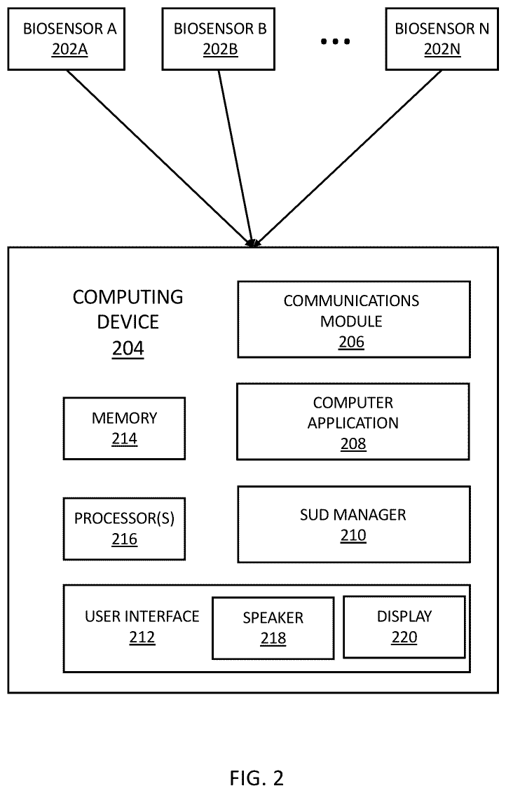

FIG. 2 illustrates a block diagram of a system, generally designated 200, for managing SUD of a person in accordance with embodiments of the present disclosure. Referring to FIG. 2, the system 200 includes one or more biosensors A-N 202A-202N (N represents that any suitable number of biosensors) for acquiring neurophysiological activity data of a person (not shown) and a computing device 202 for SUD management. Any suitable number of biosensors may be utilized for acquiring the data. Biosensor(s) 202A-202N can communicate a signal representative of the neurophysiological activity data to a computing device 204 for further processing.

With continuing reference to FIG. 2, the computing device 204 can include a communications module 206, a computer application 208, an SUD manager 210, and a user interface 212. The communications module 206 is configured to receive, from the biosensors 202A-202N, the signal representative of the neurophysiological activity data. The communications module 206 can communicate the data to memory 214 for storage. The computer application 208 can be programmed set of instructions that can reside in memory 214 and implemented by one or more processors 216 for performing tasks or functions on the computing device 204. The user interface 212 can include a speaker 218 and display 220 for implementing functions for interacting with the user, such as functions of the computer application 208 and the SUD manager 210.

The SUD manager 210 can receive the acquired neurophysiological activity data; determine that the user (or person) reached a substance abuse trigger point based on the acquired neurophysiological activity data; and operate the user interface 212 to present an interactive function of the application for distracting the person in response to determining that the person reached the subject abuse trigger point. For example, the person or user of the computing device 204 may be the person 102 shown in FIG. 102.

The computer application 208 and the SUD manager 210 can be implemented by suitable hardware, software, and/or firmware residing on the computing device 204. For example, the computer application 208 and the SUD manager 210 can be implemented by the processor(s) 216 running instructions stored in memory 214.

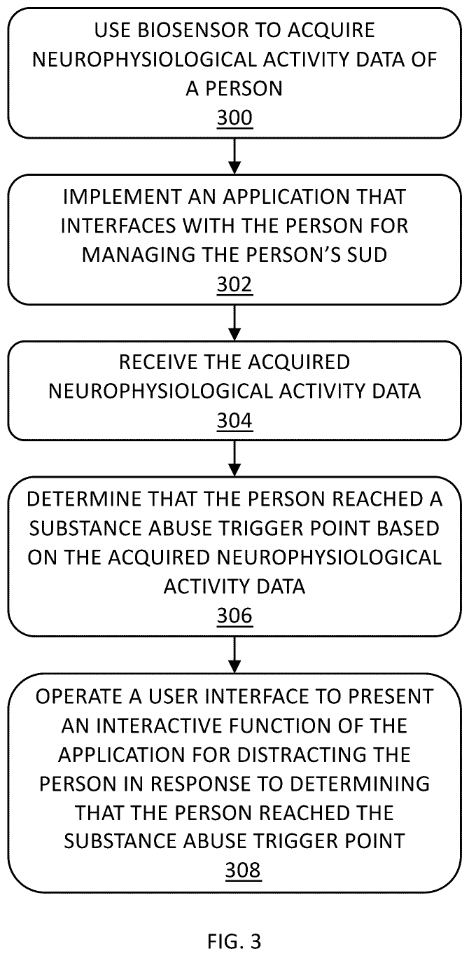

FIG. 3 illustrates a flow diagram of a method for managing SUD of a person in accordance with embodiments of the present disclosure. The method is described by example as being implemented by the computing device 204, but it should be understood that the method may alternatively be implemented by any suitable computing device.

Referring to FIG. 3, the method includes using 300 a biosensor to acquire neurophysiological activity data of a person. It is noted that although one biosensor is described as being used, multiple biosensors may be operatively attached to the person for acquiring the data of the same or different types. As an example, one or more of biosensors 202A-202N may be used for acquiring neurophysiological activity data of a person. The acquired data may subsequently be communicated to the computing device 204.

The method of FIG. 3 includes implementing 302 an application that interfaces with the person for managing the person's SUD. Continuing the aforementioned example, the SUD manager 210 is configured to initialize the computer application 208 or control the computer application 208 to perform a task or function. The task or function can prompt or cause the user to interact with the computer application 208 for distracting the person from a substance abuse activity.

The method of FIG. 3 includes receiving 304 the acquired neurophysiological activity data. Continuing the aforementioned example, the communications module 206 of the computing device 204 can received the data from biosensors 202A-202N and store the data in memory 214. The SUD manager 210 can have access to and usage of the stored data.

The method of FIG. 3 includes determining 306 that the person reached a substance abuse trigger point based on the acquired neurophysiological activity data. Continuing the aforementioned example, the SUD manager 210 can determine that the person reached a substance abuse trigger point based on the acquired neurophysiological activity data. For example, the SUD manager 210 can implement an algorithm for determining the substance abuse trigger point based on the acquired neurophysiological activity data. In examples, trigger points can be detected by determining levels of physical or emotional stress in the individual. In other examples, the SUD manager 210 can implement artificial intelligence (AI) and/or machine learning (ML) algorithms for determining when a trigger point has been met. Implementation of the algorithm includes one or more of the following factors: data acquisition, feature extraction, optimal feature selection and then stress detection and modeling. The features that we plan to use in these algorithms are EEG, EMG, EDA, and EMA feature that encompass a large set of neurophysiological and behavioral parameters that can be obtained from an individual's brain, body, or the devices they are wearing such as fitbits or smartphones. We then use the decoding and classification algorithms discussed later to identify stress and emotional states of an individual. Stress indicators can be used to determine a trigger point. For example, physiological changes can indicate that a trigger point has been reached, such as increase in heart rate, greater electrodermal activity, higher rates of respiration, and the like. Identification of these changes can allow for the onset of SUD-related stress or relapse to be detected.

The method of FIG. 3 includes operating 308 a user interface to present an interactive function of the application for distracting the person in response to determining that the person reached the subject abuse trigger point. Continuing the aforementioned example, the SUD manager 210 can operate the user interface 212 to present an interactive function of the application 208 for distracting the person in response to determining that the person reached the subject abuse trigger point. In an example, the application's interactive function can be a stimulus for distracting from substance abuse. Example functions include, but are not limited to, cognitive-behavioral therapy (CBT) interface, a neuro feedback interface, a game, or a neuro stimulation activity to the use.

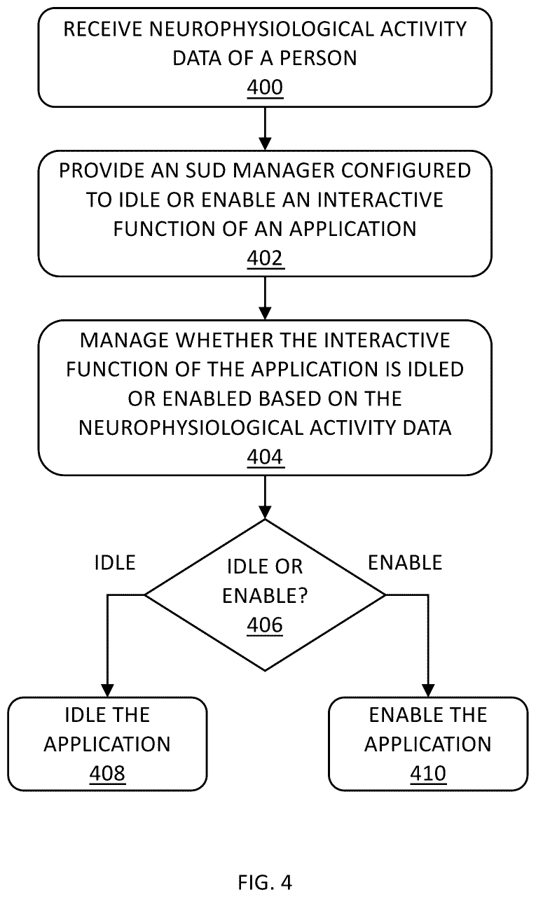

FIG. 4 illustrates a flow diagram of another method for managing SUD of a person in accordance with embodiments of the present disclosure. The method is described by example as being implemented by the computing device 204, but it should be understood that the method may alternatively be implemented by any suitable computing device.

Referring to FIG. 4, the method includes receiving 400 neurophysiological activity data of a person. For example, one or more biosensors 202A-202N can be used to acquire the neurophysiological activity data.

With continuing reference to the method of FIG. 4, providing 402 an SUD manager configured to idle or enable an interactive function of an application. For example, the SUD manager 210 can be configured to idle or enable the interactive function of the computer application 208. Selection of idling or enabling the interactive function can be based on the neurophysiological activity data.

The method of FIG. 4 includes managing 404 whether the interactive function of the application is idled or enabled based on the neurophysiological activity data. Continuing the aforementioned example, the SUD manager 210 can manage idling or enabling an interactive function of the computer application 208. At step 406, the method includes determining whether to idle or enable the application based on the neurophysiological activity data. For example, the application can be left to idle the function in response to determining that the person has not reached the subject abuse trigger point. Conversely, for example, the application can be controlled to enable the function in response to determining that the person has reached the subject abuse trigger point. At step 408, the method includes idling the application in accordance with that determination. At step 410, the method includes enabling the application in accordance with that determination.

In embodiment, different functions can be implement depending on a trigger point level determined for the person. Example trigger point levels include, but are not limited to, the categories of mild, moderate, and severe. In the case of a mild level, a cognitive-behavioral therapy (CBT) interface can be presented to the person via a user interface. In the case of a moderate level, a neuro feedback interface (e.g., videogames) can be presented to the person via a user interface. In the case of a severe level, a neuro stimulation activity can be provided to the person via a user interface.

In accordance with embodiments, models are provided for indicating substance abuse trigger points. The models involve using measurements of physiological biomarkers tracked throughout various emotional states. In studies, two types of mental stress and social stress were induced, which are also induced by SUD. In order to identify biomarkers (or triggers) that indicate stress, tracked features were collectively analyzed through a multi-way classification, reflecting the various emotional and stress levels that one may experience in daily life. Additionally, models were trained to identify whether a mental or social stressor is being experienced by the subject at a given moment. This differentiation of the type of stress can be used for determining which prevention or intervention method may be the most efficient and effective.

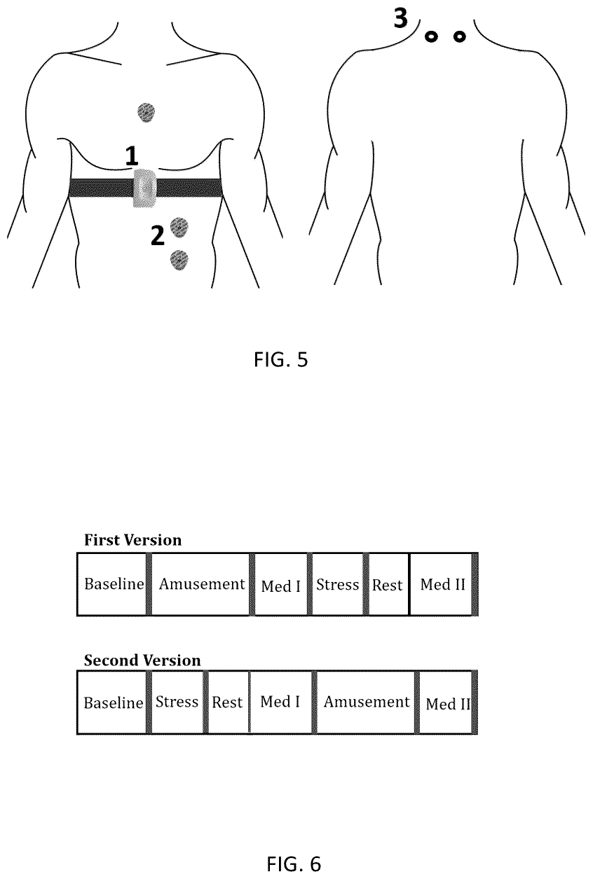

In experiments, a multimodal dataset for Wearable Stress and Affect Detection (WESAD) was used to study the physiological changes and responses to stress induced by SUD. These changes in the body were tracked using two wearable devices: RespiBAN Professional, which is worn around the chest, and Empatica E4, which is worn around the wrist. Embedded in these devices were sensors to track three axis accelerometers on the chest (X, Y, Z), electrodermal activity (EDA), electromyograph (EMG), respiration (RESP), electrocardiogram (ECG), and body temperature (TEMP); depicted in FIG. 5, which illustrates a diagram showing RespiBAN Professional's placement of electrodes (1. RespiBAN Professional with temperature, EDA, and control module. 2. Three ECG electrodes. 3. Two EMG electrodes on the back where the shoulder meets the neck). However, for the purpose of a computational analysis on identifying the features that can best indicate stress, only the recordings of RespiBAN Professional were used. This was chosen over Empatica E4 due to its larger volume of data points available (over 2 million data points per subject versus 20,000-50,000 data points per subject).

The original data collection was conducted for 17 subjects in total, but due to sensor malfunction, the data was only available for 15 subjects. Out of these remaining subjects, 12 were male and 3 were female, and they had a mean age of 27.5±2.4 years. According to the study protocol, four emotional states—baseline, stress, amusement, and meditation—were induced in stages within all participants (see e.g., FIG. 6). FIG. 6 are depictions of two protocols tested under the study. The bars indicate the times when the study participants filled out questionnaires for self-report. Descriptions of these states and how they were induced are described below.
Baseline: A neutral state was induced as subjects sat or stood at a table and read neutral material.
Stress: A highly strenuous state was induced in which subjects were exposed to both parts of the Trier Social Stress Test:
   Mental stress: a mental arithmetic task.
   Social stress: a public speaking task.
Amusement: An amusing state was induced as subjects were shown funny video clips.
Meditation: A de-excited state was induced as subjects were guided through meditation exercises.

At the end of each state, the participants filled out a questionnaire that asked them to rate different emotional states. The questionnaire for all states included the Positive and Negative Affect Schedule (PANAS), the State-Trait Anxiety Inventory (STAI), and Self-Assessment Manikins (SAM) tests. The stress state had an additional Short Stress State Questionnaire (SSSQ). The ratings assigned within each of these tests were utilized as a standard ground truth to assess the validity of the stress detection models proposed.

To perform the exploratory and predictive analyses on the data, we used MATLAB® Machine Learning Toolbox and Python. The Python libraries used included pandas, sklearn, matplotlib, and numpy. Specifically, the sklearn library provided the tools to implement various algorithms. The integrated development environment (IDE) used was Jupyter Lab in combination with the Anaconda platform.

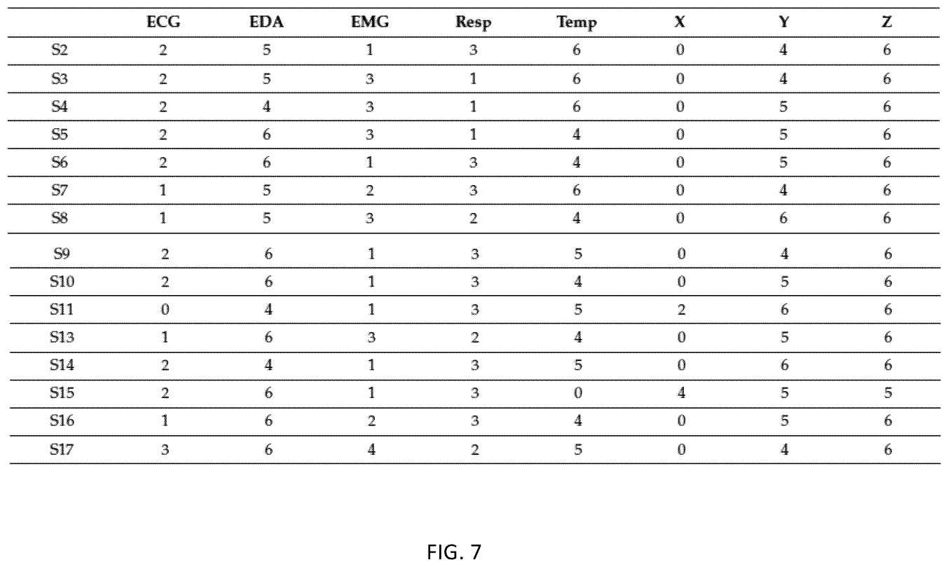

In order to preprocess the missing values in the dataset, values were first substituted based on the mean or mode of the distribution. Then, the data was normalized to have optimal distribution, so that no value drove the model's performance in one direction or skewed the prediction. All values had equal weightage and statistical importance in the dataset as a result of this. The physiological biomarkers that were the best indicators of accurate stress detection were identified from the dataset through logistic regression, linear regression, and principal component analysis (PCA). Additionally, sequential forward feature selection utilizing quadratic discriminant analysis was conducted for the purpose of a feature analysis, as presented the table shown in FIG. 7, which shows the significance of these features.

In experiments, three types of classifications were performed. They were as follows:
(a) 2-way: stress vs. amusement;
(b) 3-way: stress vs. amusement vs. meditation;
(c) 4-way: stress vs. amusement vs. meditation vs. baseline.

In other experiments, three different types of classification were performed in order to differentiate social stress and mental stress, and to also explore the effects of meditation on stress:
(a) 3-way: baseline vs. meditation before stress vs. meditation after stress;
(b) 3-way: baseline vs. social stress vs. mental stress;
(c) 6-way: baseline vs. social stress vs. mental stress vs. amusement vs. meditation before stress vs. meditation after stress.

These classifications for predictive analysis were performed using three approaches:
(a) logistic regression;
(b) decision trees;
(c) XGBoost (gradient-boosted decision trees).

K-fold cross-validation was implemented and the results of the predictive analysis were measured using accuracy and area under the curve as performance metrics. While accuracy is a standardized and commonly-used metric, it is also crucial to meticulously calculate the accuracy that could not be achieved. This will help to minimize false positives. Hence, for binary classification, also analyzed was the area under the curve to check the degree of separation between true positives and false positives. The ratio of trained and test data split was 4:1.

Here, the three types of classifications were performed and the rationale behind these classifications provided: (a) 3-way: baseline vs. meditation before stress vs. meditation after stress; (b) 3-way: baseline vs. social stress vs. mental stress; and (c) 6-way: baseline vs. social stress vs. mental stress vs. amusement vs. meditation before stress vs. meditation after stress.

3-Way: Baseline Vs. Meditation Before Stress Vs. Meditation after Stress

There are two versions of this classification, depending on the time of occurrence of rest. In the first version, the sequence of events is as follows: Baseline->meditation before stress->rest->meditation after stress. For the second version, the sequence of events is as follows: Baseline->rest->meditation before stress->meditation after stress. In the study, different subjects were subjected to different versions and no subject was subjected to both versions. The aim of this classification was to compare how meditation before and after stress affects the human body. For the first version, the technique to find the exact data point where the meditation before stress begins was by finding the data point for the last occurrence of amusement and first occurrence of stress. The data points in between these two conditions represent meditation before stress, or ‘Med I’, as shown in FIG. 6.

Meditation after stress was computed by locating the last data point for stress and then picking up the remaining data points for stress. Thereafter, the data points for both these types of meditation were combined with the data points for baseline to form a new dataset, which was then used for predictive analysis using the aforementioned algorithms and metrics in Python for each subject. To attain a better understanding of the features that contributed the most, a forward feature selection technique using quadratic discriminant analysis was implemented and the top three contributing features were extracted for each subject. These were then used as inputs to the predictive analysis algorithms used to study the results.

Similarly, the data points for meditation before and after stress for version 2 were extracted. To find the data points for meditation before stress, the last data point representing rest was located and the first data point representing amusement was located. The data points in between these represent meditation before stress for version 2, as shown in FIG. 6. The data points representing meditation after the last occurrence of amusement were labeled as meditation after stress. These extracted data points were then combined with baseline data to form a new dataset, which was then subjected to predictive analysis using the above three algorithms for classification, and independently judged using accuracy and area under the curve as metrics for all subjects. Thereafter, using forward feature selection and quadratic discriminant analysis, the top three contributing features were extracted, and the same predictive analysis procedure was implemented.

3-Way: Baseline Vs. Social Stress Vs. Mental Stress

All the subjects that participated in this experiment were subjected to two types of stress: mental stress and social stress. Mental stress was observed while computing a mathematical problem or counting down numbers, whereas social stress was observed during public speaking. The WESAD dataset represents these stress types with only one label. However, every subject was first subjected to social stress first and then mental stress. The duration of the two types of stress, and therefore, the number of data points for each of these stress types, was the same. This allowed us to correctly label the first half of the stress data points as social stress and the second half as mental stress. After extracting these data points, a new dataset was formed that consisted of baseline, social stress, and mental stress. This dataset was then used as an input to a predictive analysis model for classification using logistic regression, decision trees, and XG-Boost algorithms, and then this performance was judged using accuracy and area under the curve as the classification metrics for each subject. Furthermore, using forward feature selection and quadratic discriminant analysis, the top three features were extracted from the above-formed dataset and then independently subjected to the same classification for every subject as above.

6-Way: Baseline Vs. Social Stress Vs. Mental Stress Vs. Amusement Vs. Meditation Before Stress Vs. Meditation after Stress

For this predictive model, we combined the above two datasets in accordance with the versions that the subjects were exposed to. Additionally, the amusement labels were also appended to the dataset. The sequence of events for the first type of classification (or version 1) are as follows: Baseline->Amusement->Meditation before stress->Social Stress->Mental Stress->Rest->Meditation after stress. The sequence of events for the second version are as follows: Baseline->Social Stress->Mental Stress->Rest->Meditation 1->Amusement->Meditation 2.

First, a simple classification was performed using predictive analysis that was implemented using logistic regression, decision trees, and XG-Boost algorithms, and then the performance was measured using accuracy and area under the curve for each subject separately. Thereafter, the top three contributing features were extracted using forward feature selection and quadratic discriminant analysis. Again, these features were used by the same three algorithms and measured using the same classification metrics.

Results of experiments are indicative the top biomarkers. The accuracies produced by each statistical test and combination of features were specified and accordingly ordered. To calculate the most significant combination of physiological features, our first step was to implement sequential forward feature selection using quadratic discriminant analysis. For every candidate, we assessed which would be the most significant 2, 3, 4, 5, 6, and 7 features. The table in FIG. 7 depicts the findings from this analysis, as well as all the features plotted against the corresponding candidates. The numerical value signifies the number of times the features were chosen in combination with other features for the same subject. On a scale of zero through six, zero implies the non-existence of that feature in any combination conducted for that subject (least relevant), while six indicates the occurrence of that feature in all combinations carried out for that subject (most relevant). A total of six iterations were performed with various feature counts ranging from a combination of two features to a combination of eight features. This selection process was different for different subjects.

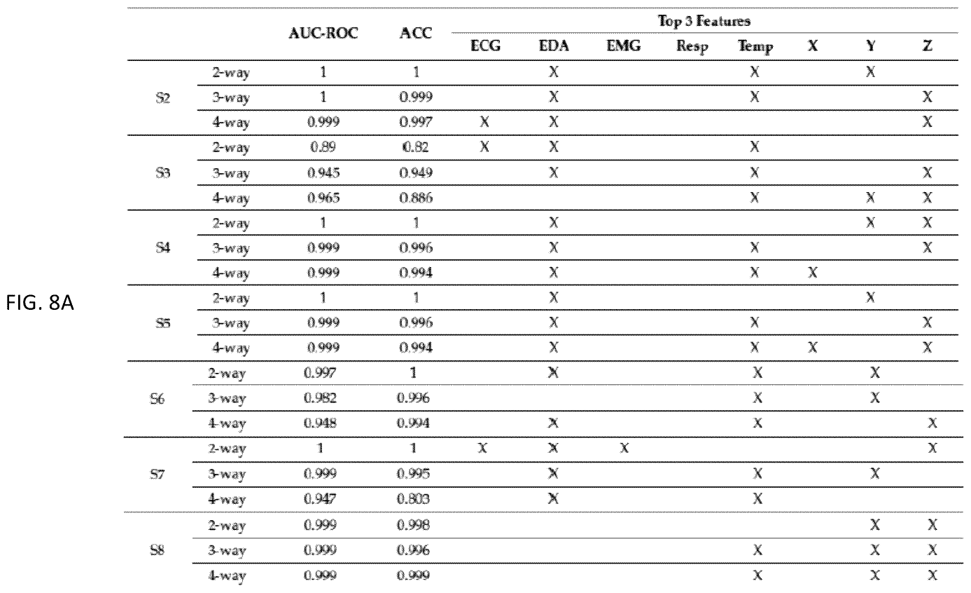

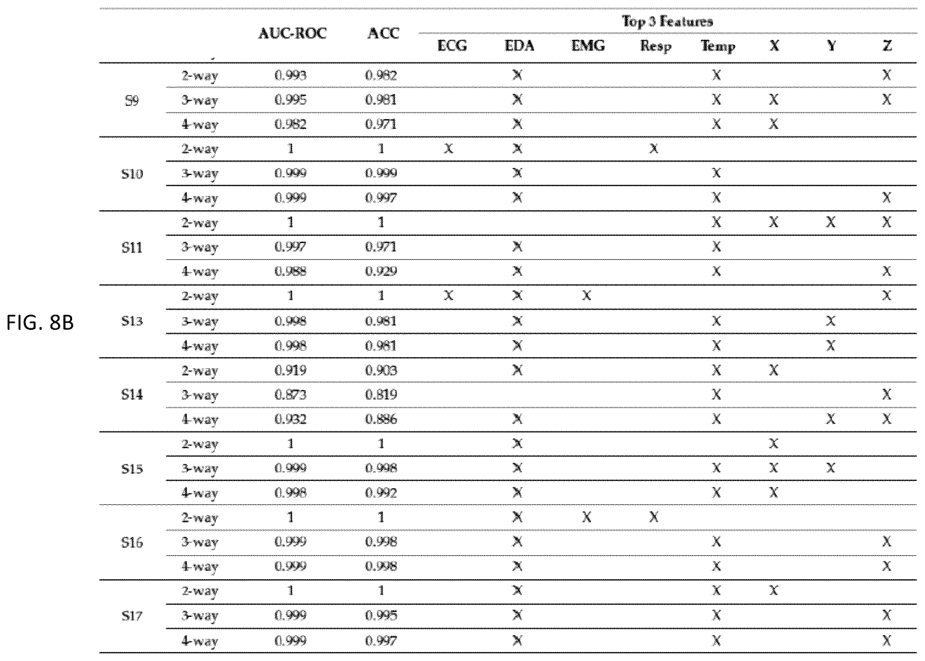

It was observed that the most used and considered feature in every combination for all but one of the 15 subjects was the accelerometer Z-axis (Z), due to its direct association to heartbeat. The feature that was second-most used and included in all six cases for four of the candidates was EDA. On the other end of the spectrum, the least used and the least significant feature used in none of the combinations for 13 subjects was the accelerometer X-axis (X). However, since the accelerometer, as a whole, contributed to the classification accuracy, this variable cannot be ignored for any of the future studies. Furthermore, to identify and validate features that could best predict stress, a feature analysis with logistic regression and PCA was performed for every subject. This helped us discover several other features that emerged as being more important than the rest. The features that had a strong association to a subjects' mental and emotional states were EDA and temperature, along with the accelerometer (z-axis). The outcomes attained from logistic regression with 2—(stress vs. amusement), 3—(stress vs. amusement vs. meditation), and 4-way (stress vs. amusement vs. meditation vs. baseline) multivariate classification are displayed in the table shown in FIGS. 8A and 8B, which shows classification accuracy using logistic regression with top three features. In general, logistic regression had an average accuracy (ACC) of 0.969 and an average AUC-ROC of 0.985 in all the subjects.

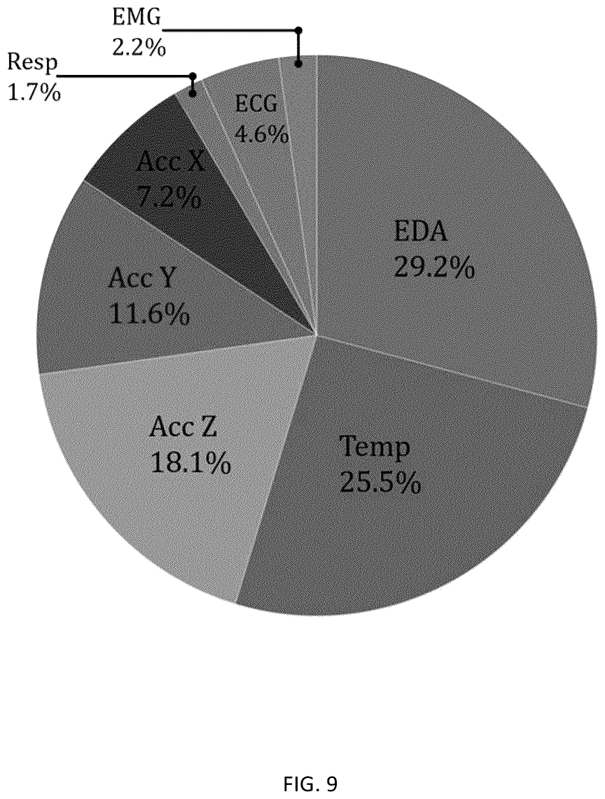

In addition to logistic regression, similar classification was repeated using the Decision Tree method. Not only did the Decision Tree choose the same features discovered from the logistic regression (EDA, temperature, and the accelerometer z-axis), but it also worked slightly better, with an average AUC-ROC of 0.998 and an average accuracy of 0.968. To validate the results, another classification algorithm, XGBoost, was used. This classifier also chose the EDA, temperature, and the accelerometer z-axis features, and it outperformed both the logistic regression and the Decision Tree classification algorithms in the classification accuracies. In all, a total of 135 individual tests were run-45 test runs with each of the 3 classifiers. Of these 135 tests, EDA was chosen as one of the top three features 121 times, making it the most significant feature. The second-most significant feature was temperature, as it was chosen 106 times as a top feature. Finally, the accelerometer z-axis was chosen as the third-most significant feature, due to being one of the top three features 76 times. These features, along with others, are depicted in FIG. 9, which illustrates a pie chart showing significance of the features based on how frequently they were used in three different classifiers for best accuracies. In FIG. 9, EDA, temperature, and accelerometer Z (and Y) stand out as the important features. Detecting whether the subject is stressed depends on a combination of various factors, so finding a relevant combination of pertinent features was a vital task. Selecting only one feature would probably not provide relevant and generalizable results.

In summary, we found that the predictive models performed better when supplied with the top three contributing features as inputs, instead of all the features. Logistic regression yielded lower accuracy and AUC score as compared to Decision Trees and XGBoost. XG-Boost showed an overall excellent performance in most of the subjects. High performance was achieved even after considering six types of labels (6-way classification) by selecting the best combination of parameters for the predictive analysis models. In our limited experience with the data, the accelerometer was the most common feature in all subjects for all three types of classifications. Furthermore, we were able to effectively distinguish between mental stress and social stress, and thereby, create two new sub-labels which will be very useful in future studies.

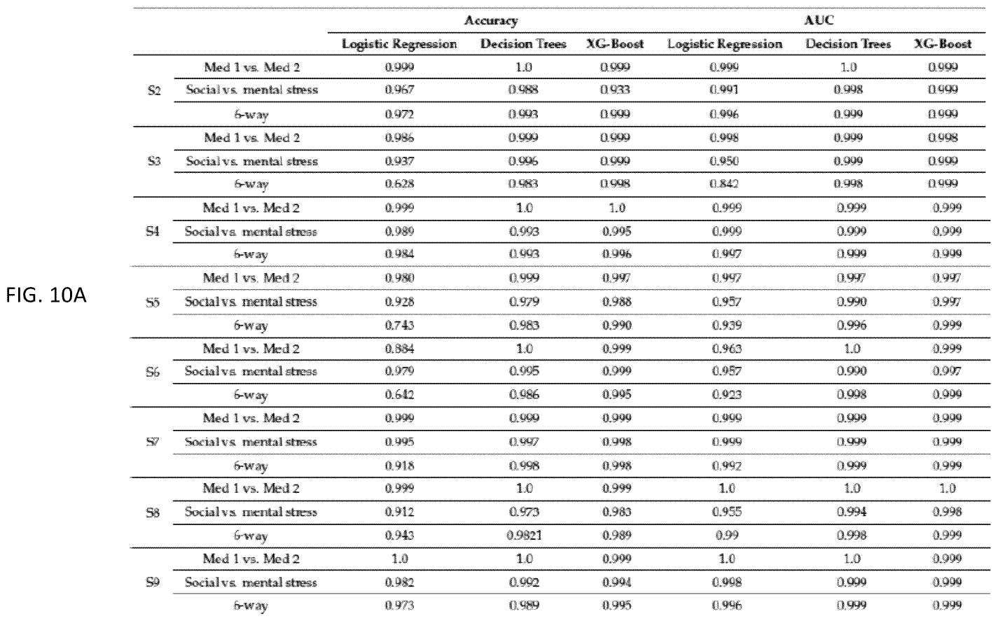

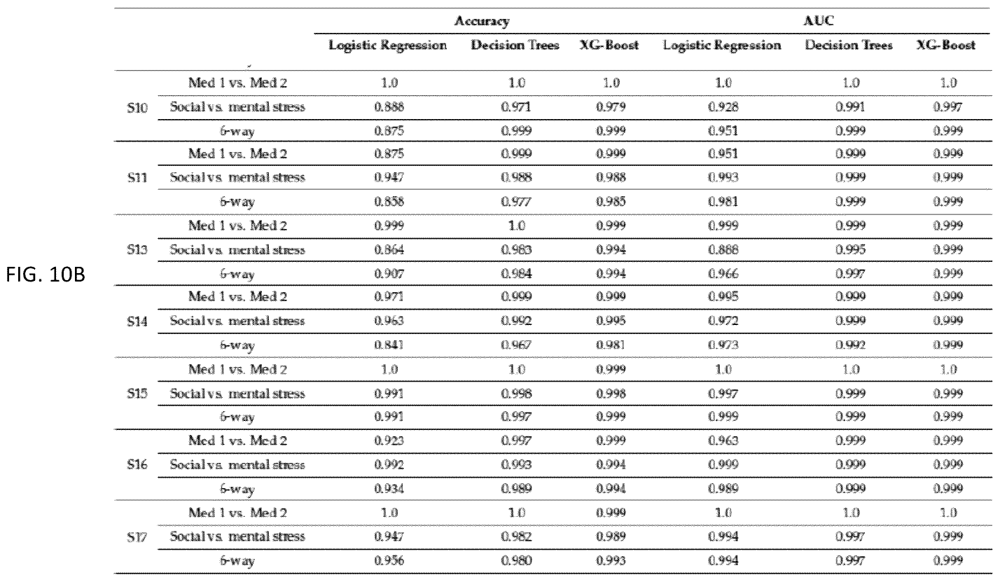

The table shown in FIGS. 10A and 10B depicts accuracies and AUC for all types of classifications for all subjects using three algorithms and all features.

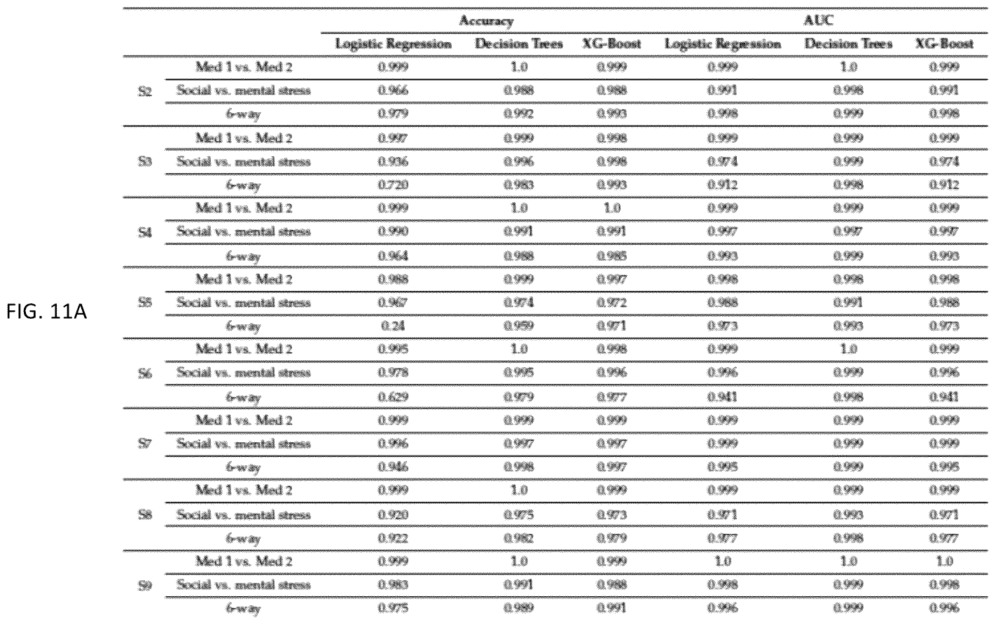

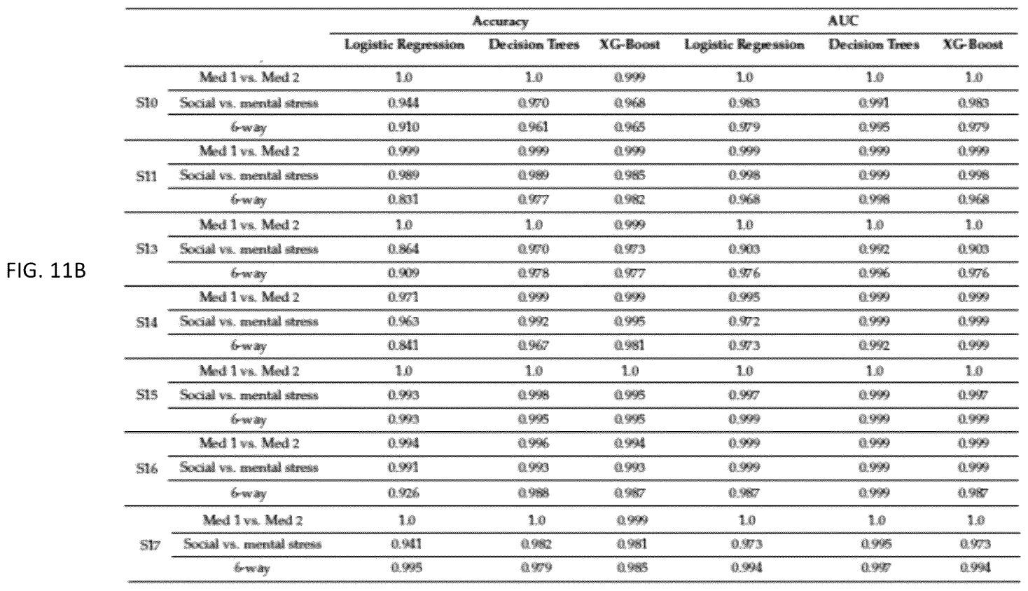

The table shown in FIGS. 11A and 11B depicts accuracies and AUC for all types of classifications for all subjects using three algorithms and the top three features.

An objective of these studies was to identify optimal biomarkers that can best assist in the accurate detection of stress. It was found that EDA, body temperature, and chest-worn accelerometers are important features in stress detection. It was aimed not to find just one best feature, but a combination of multiple features that can help in precise detection as the fusion of multimodal features improves the detection of accuracy. As shown in the table of FIGS. 10A and 10B, optimal features change from subject to subject, and this enabled us to build personalized models that were unique to individuals. Additionally, we were able to train our models to identify which type of stressor, mental or social, was being experienced based on the various administrations of the experiment. This analysis can help researchers better identify the triggers for relapse and recommend informed and appropriate treatments.

These studies also provided a multi-way classification to identify the fusion of sensors that would provide the best indication of stress. The stressor type was analyzed and identified. Additionally, various administrations of the tests were used, with varying orders of states in which the subjects were in, which allows for us to identify which states the person must be in order for the classifier to most accurately identify stress. Together, these goals allowed our group to focus on the aspects most prevalent in SUD-related stress and information potentially useful for treatment.

The WESAD dataset, having been openly available online since 2018, has been used in many other studies with similar goals—the detection of stress. Research closest to the one that our group conducted involved the identification of stress and stressor type through various machine learning classifiers and models. Since the dependent variable of the state of the participants was binary, a logistic regression model was used over a linear regression model. This model identified respiration rate as the strongest indicator of stress, along with the combination of respiration rate, heart rate, and heart rate variability, providing an accuracy of 85.70%.

Even though an aim was to detect stress as a trigger in the context of substance abuse, the methods outlined and developed in this paper have several applications in various domains, as stress can lead to multiple other complications. Stress negatively affects cognitive functions, weakens memory, increases blood pressure, and causes cardiac disorders and diabetes, to list a few, especially as instances of acute stress begin to build up in degree and instance, and can eventually develop into chronic stress among other, even more dangerous illnesses. In each of these diseases and disorders, stress manifests in different ways, reiterating the benefit of the multimodal fusion of features in improving the detection accuracy across domains.

Decades of research have shown that stress increases risk of substance abuse, and could be a hindrance to effective treatment of substance abuse. The use of substances is known to stimulate the release of the neurotransmitter dopamine, providing an intense pleasurable feeling which creates a positive feedback loop within the user. This leads to the uncontrolled use of illicit drugs, alcohol, excessive use of legal drugs, or other addictive behaviours. Further negative effects of SUD can manifest themselves in substance users in the form of stress, among other symptoms. The detrimental cycle of addiction has the power to significantly impair the lives of users in terms of their decision-making skills, ability to meet responsibilities at school and/or work, personal relationships, and internal well-being. One's physical dependence on a substance to get through these daily activities and experiences makes stepping away from a certain substance psychologically stressful. This may lead to relapse or Post-Acute Withdrawal Syndrome, a prolonged experience of withdrawal symptoms. When these instances of stress begin to add up or when their symptoms are prolonged, the acute stress measured in the experiment can develop into chronic stress. Additionally, any instance of acute stress can provoke a heart attack or a stroke. This connection between the two forms of stress provides for a better analysis for stress detection and management in substance users, since the physiological symptoms are likely to appear in the same way.

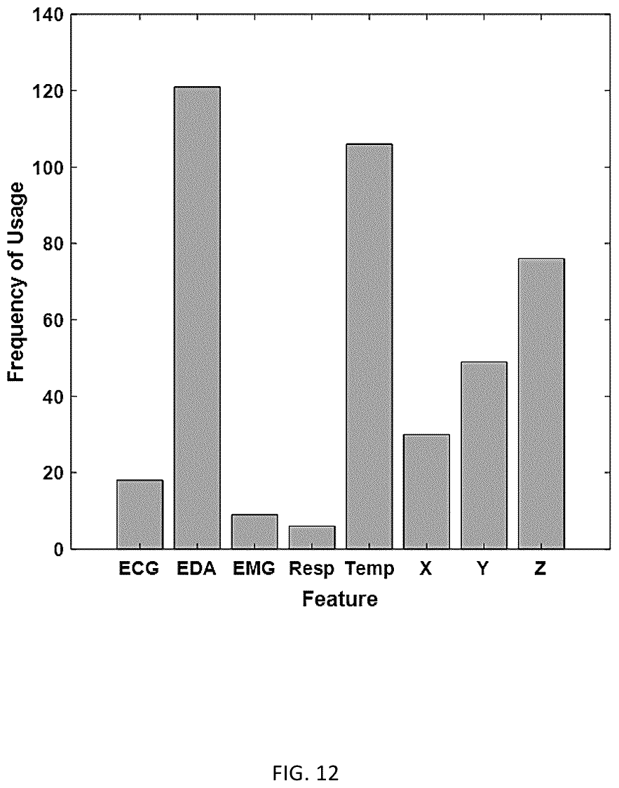

FIG. 12 illustrates a graph showing significance of the features based on how frequently they were used in three different classifiers for best accuracies. EDA, temperature, and accelerometer Z stand out as important features.

In other experiments, six healthy subjects (one female, average age 23.7 years, range 18-29 years) participated in this study. Two protocols were conducted in this study, the Montreal Imaging Stress Task (MIST) (Dedovic et al., 2005) and the Trier Social Stress Test (TSST) (Kirschbaum et al., 1993), to stimulate mental stress and social stress respectively. After each section, a self-assessment questionnaire was filled out by the participant to evaluate their stress level scoring from 0-4 representing not stressed at all to extremely stressed. Between each protocol, 20 minutes of break was provided.

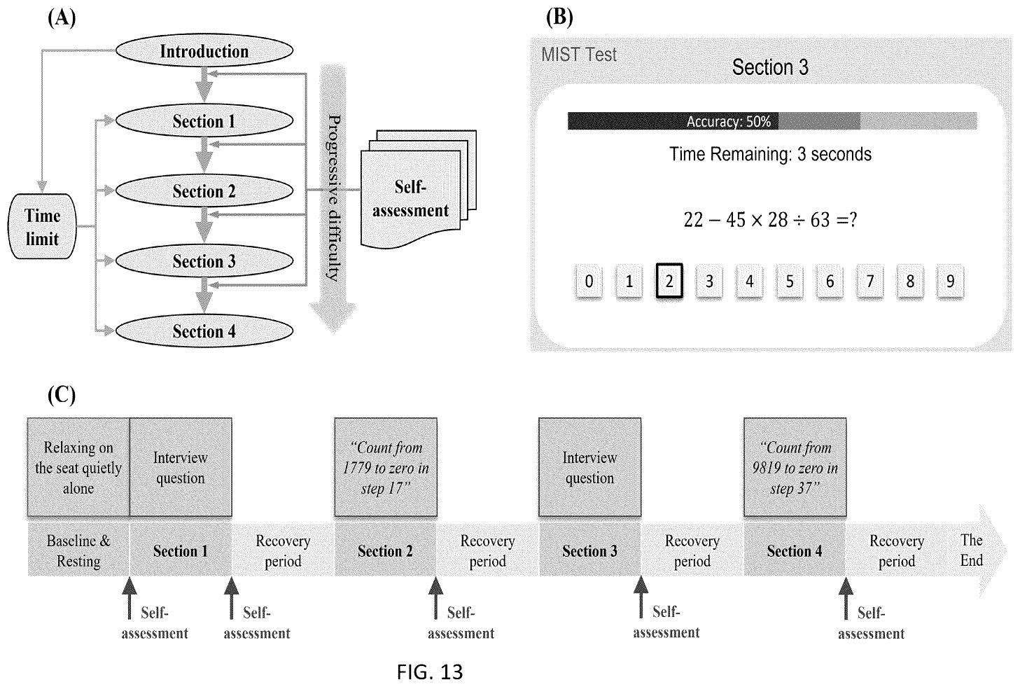

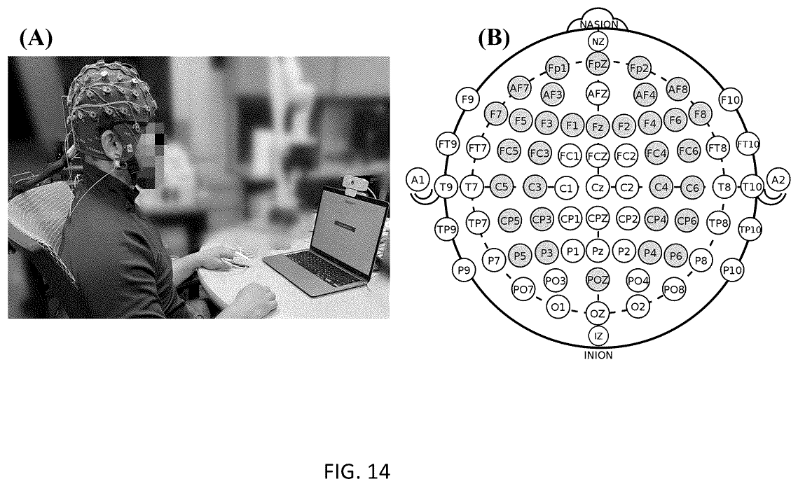

The MIST protocol consists of five sections of arithmetic tasks, including an Introduction section and four progressively challenging test sections. FIG. 13 illustrates a diagram of an example MIST paradigm. Each section has a duration of three minutes. The Introduction section illustrates sample math tasks, recording the time taken for each question. An average time was computed from the Introduction section to establish a time limit for the subsequent four test sections. In the test sections, participants were presented with analogous math tasks but were subject to time constraints and received stressful auditory and visual feedback for incorrect/missing calculations. Key elements of the MIST interface, as shown in FIG. 13 part (B), include a central display of the math question, response options located below, a countdown displayed above, and a progress bar at the top of the screen indicating the accuracy percentage. The accuracy bar transitions from green to red if accuracy falls below 60%. Each question is assigned a time limit based on the participant's performance in the Introduction section. Upon providing a correct response, participants receive a subtle auditory cue along with a visual indicator, denoting “correct” before transitioning to the subsequent question. Conversely, an incorrect response or a lack of response prompts an auditory alert coupled with the display of a conspicuous pop-up window conveying “Incorrect” and “Time is out,” respectively. No meditation or rest periods were provided between sections, to allow for the observation of the expected increase in stress levels.

TSST protocol incorporates two designated speaking sections with interview questions and two sections of mental arithmetic tasks, with a duration of three minutes for each section. In the speaking sections, participants were presented with questions and required to respond without any preparation time. Meanwhile, the participants were expected to maintain eye contact with the instructor and sustain their speech until the allocated time concluded. In the arithmetic sections, the participants were requested to count from a four-digit number to zero in increments of a two-digit prime number. Participants were required to restart the counting process upon making any mistakes. Subsequent to each section, participants were afforded a ten-minute rest period to recover. The procedure is illustrated in FIG. 13 part (C).

The experiment conduction and data collection were under an approved protocol by the Internal Review Board (IRB) of the University of Maryland Baltimore County. Before the stress experiment, the subjects were sitting and relaxing at the experimental table with three minutes of baseline recorded (FIG. 2(A)). During the experiment, electrophysiological signals and EEG signals were measured and recorded with a g.tec Amplifier (g.HIamp, g.tec medical engineering GmbH, Graz Austria)) at a 512 Hz sampling rate, low-pass filtered at 30 Hz. The pulse plethysmogram (Pleth), SpO2 and heart rate (HR) were measured using a pulse oximeter sensor (EnviteC) clipped on the index fingertip. The galvanic skin response (GSR) or electro-dermal activity (EDA) records the electrical conductivity using the two g. GSRsensors placed on the middle and ring fingertips. The temperature was recorded by g. TEMPsensor within a range of 20-45° C. placed on the palm. In order to allow the subjects to do the tasks with their dominant hand, all electrophysiological sensors were placed on their non-dominant hands. For EEG acquisition, a high-density EEG cap based on 10/20 system position was used to collect neural signals from 33 active EEG electrodes located at Fp1, Fpz, Fp2, AF7, AF3, AF4, AF8, F7, F5, F3, Fz, F2, F4, F6, F8, FC5, FC3, FC4, FC6, C5, C3, C4, C6, CP5, CP3, CPZ, CP4, CP6, P5, P3, P4, P6, Oz (FIG. 2(B)). During the EEG signal recording, the subjects will be instructed to minimize head movement during the three-minute collection.

Each three-minute data section was initially segmented into shorter lengths for analysis. To explore the potential for real-time stress detection with minimal delay, we examined four signal durations: 60 seconds, 30 seconds, 10 seconds, and 4 seconds. To effectively preserve information while using shorter segments, each duration was extracted from the original recordings with a 50% overlap. The physiological features were extracted from each segment and a detailed description of these features is given below.

HR, SpO2 and temperature: Mean and standard deviation (std) were calculated within each segment.

GSR/EDA is one of the body responses to stress and is widely used for quantifying the autonomic nervous system (ANS) activity. As a part of the response, the sweat from the body surface leads to changes of skin conductivity, indicating the changes in the skin's electrical characteristics due to perspiration, reflecting stress variations. When a person becomes more or less stressed, the GSR increases or decreases respectively. The time series of GSR comprises two fundamental components. One is the skin conductance level (SCL) which relates to the slower-acting components and background characteristics of the signal reflecting general changes in autonomic arousal. The other is the skin conductance response (SCR) which refers to the rapid short-lasting changes in the GSR caused mainly due to neuronal activity (Ghosh et al., 2015). First, the GSR signals were decomposed into phasic components (SCR) and tonic components (SCL) using nonnegative deconvolution by solving a convex optimization approach problem using the Matlab function cvxEDA. The mean, std, maximum and minimum values were calculated as features from these two components respectively. The SCR higher than threshold 0.1 was considered as effective response, and the corresponding number of responses, maximum and minimum distance between two responses were extracted as features.

PPG/Pleth displays the volumetric change of blood in the vessels according to the respiratory cycle. A standard time domain heart rate variability (HRV) and pulse rate variability (PRV) can be reliably derived from ultra-short term (60 s) PPG recording. HRV, the temporal fluctuation of consecutive heartbeats, is discerned through the pulse wave signal generated by a PPG sensor. The pulse cycle intervals (NN intervals) extracted from this signal present a compelling alternative to ECG for HRV analysis, often referred to as (PRV) analysis.

Time domain features were computed including:
Beats Per Minute (BPM): An essential metric used to measure pulse rate.
Mean of PRV Time Elapsed Between Successive Normal PPG Onset Intervals (meanNN) represents the average time between two successive normal PPG onset intervals, also considered as normal R-waves in HRV. Measured in milliseconds, this metric contributes to understanding the temporal dynamics of PRV.
Standard Deviation of NN Intervals (SDNN) quantifies the standard deviation of NN intervals. An SDNN value greater than 50 ms suggests normal stress, providing valuable information about the variability in consecutive heartbeats.
Percentage of Successive Beat-to-Beat Intervals >50 ms (pNN50) reflects the percentage of successive beat-to-beat intervals that differ by more than 50 ms. A decrease in pNN50 percentage suggests lower parasympathetic activity, offering insights into the autonomic nervous system's modulation.
Root Mean Square of Successive NN Interval Differences (RMSNN) is an important metric for estimating high-frequency variations in pulse rate, particularly pertinent for short-term RR recordings. RMSNN contributes to understanding the intricate dynamics of consecutive heartbeats.

Time domain features were computed including:
Beats Per Minute (BPM): An essential metric used to measure pulse rate.
Mean of PRV Time Elapsed Between Successive Normal PPG Onset Intervals (meanNN) represents the average time between two successive normal PPG onset intervals, also considered as normal R-waves in HRV. Measured in milliseconds, this metric contributes to understanding the temporal dynamics of PRV.
Standard Deviation of NN Intervals (SDNN) quantifies the standard deviation of NN intervals. An SDNN value greater than 50 ms suggests normal stress, providing valuable information about the variability in consecutive heartbeats.
Percentage of Successive Beat-to-Beat Intervals >50 ms (pNN50) reflects the percentage of successive beat-to-beat intervals that differ by more than 50 ms. A decrease in pNN50 percentage suggests lower parasympathetic activity, offering insights into the autonomic nervous system's modulation.
Root Mean Square of Successive NN Interval Differences (RMSNN) is an important metric for estimating high-frequency variations in pulse rate, particularly pertinent for short-term RR recordings. RMSNN contributes to understanding the intricate dynamics of consecutive heartbeats.

Frequency domain measures of short-term HRV estimated the distribution of power into low-frequency band (LF, 0.04-0.15 Hz) and high-frequency band (HF, 0.15-0.4 Hz) and total frequency band (0-5 Hz). LF bands which are modulated by the sympathetic and parasympathetic nervous systems. In this study the computed frequency domain features include HF power, LF power, LF/HF (LF power to HF power ratio), LF power to total power ratio and HF power to total power ratio.

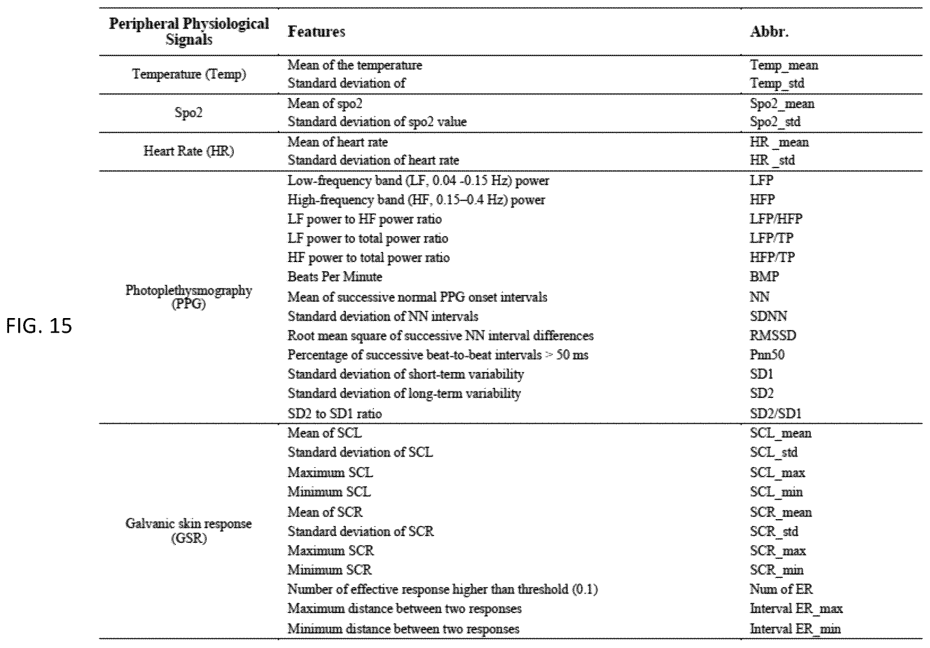

Furthermore, Poincare parameters provide a geometrical method for analyzing HRV and represent the correlation between successive inter-beats intervals. SD1 and SD2 are used to qualify Poincare plot geometry, where SD1 is the standard deviation of short-term variability and SD2 is the standard deviation of long-term variability, as illustrated below.

$$SD 1 = \frac{\sqrt{2}}{2} std ( {x}_{i} - {x}_{i + 1} )$$

$$SD 2 = \sqrt{2 {std ( {x}_{i} )}^{2} - \frac{1}{2} {std ( {x}_{i} - {x}_{i + 1} )}^{2}}$$

where std is the standard deviation of the HRV time series and x represents the time interval (i) between successive beats. A sufficient increase in SD2/SD1 in the presence of stimulus resulted in a decrease in total PRV and indicates that the subject is stressed. The detailed features are illustrated in the table shown in FIG. 15, which is a table showing data of features extracted from peripheral physiological signals.

During the three-minute data collection, EEG signals contain a lot of noise and artifacts. First, the polynomial trends were removed from the raw EEG by subtracting the evaluated polynomial curve. To eliminate the eye movement artifacts, an algorithm based on discrete stationary wavelet transform (DSWT) combined with independent component analysis (ICA) was designed to identify and remove ocular artifacts from EEG signals. The artifact removal based on the ICA-DWT technique has been demonstrated as an effective technique in EEG data cleaning. The EEG signals were first decomposed into approximation and detail coefficients under 5-level decomposition using DSWT. After the ICA was applied to remove ocular artifacts from the approximation coefficients, the cleaned EEG signals were reconstructed then using inversive DSWT.

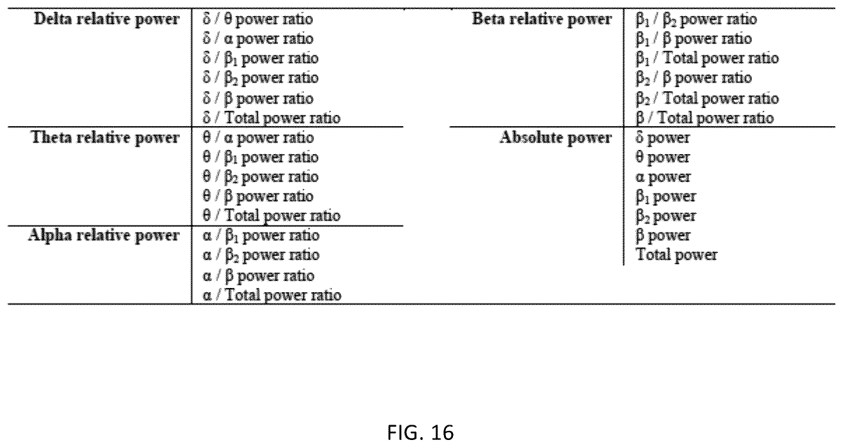

Since the physiological and EEG signals were pre-filtered to 0-30 Hz during the data collection, the EEG signals were divided into five rhythms including delta (8, 1-4 Hz), theta (0, 4-8 Hz), alpha (α, 8-218 13 Hz), beta I (β₁, 13-22 Hz), beta II (β₂, 22-30 Hz), and broad range (1-30 Hz). The spectral powers 219 of EEG signals from each rhythm were estimated within the whole time period. In addition to the 220 spectral powers from each frequency band, the power ratios across the different rhythms were 221 considered as main features from each channel of EEG signals. The full lists of all features are given 222 in the table shown in FIG. 16, particularly features extracted from EEG signals.

In order to investigate the detectability of stress levels and the discernment of stress types based on physiological and EEG signals, it was imperative to address the individual variability in physiological responses to stressors. Thus, stress states were discerned within each subject, accounting for the idiosyncratic changes in physiological features during stress-inducing events.

Preceding the application of classifiers, a meticulous data preprocessing step was executed. This 229 involved the removal of trials containing inf or NaN values from the dataset. Subsequently, an 80%-20% split was performed for each class, designating 80% of the trials as the training set and the remaining 20% as the testing data. To assess the efficacy of the employed classifiers, a robust 10-fold cross-validation methodology was employed, ensuring the selection of distinct batches of training and testing data in each fold.

In the field of stress studies, a variety of classifiers have been explored for the assessment of mental stress levels. This study specifically opted for the inclusion of well-established and widely recognized classifiers, including Support Vector Machines (SVM), K-Nearest Neighbors (KNN), Naïve Bayes (NB), Linear Discriminant Analysis (LDA), and Decision Tree (DT). These classifiers were strategically chosen to facilitate multi-level stress detection and the nuanced identification of various stress types.

In exploring the efficacy of stress detection utilizing both physiological and EEG signals for discerning stress levels and types, individual analyses of physiological and EEG signals were conducted to assess whether the combined approach enhances the discriminatory performance in stress detection.

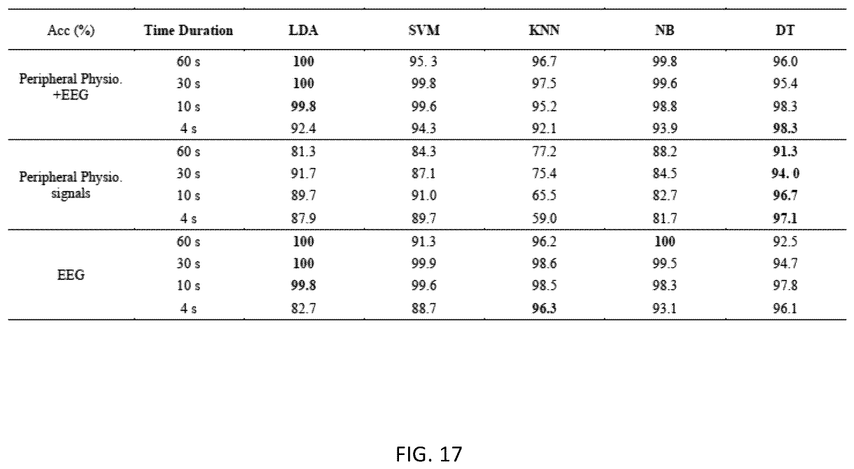

The table shown in FIG. 17 presents a comprehensive comparison of classification accuracy across various approaches for discriminating non-stress, social stress, and mental stress using different classifiers. In stress detection exclusively utilizing physiological signals, the Decision Tree emerged as the predominant classifier, surpassing all others and achieving remarkable average accuracy across all studied time lengths.

Remarkably, the classification performance highlights that a shorter period of time leads to superior stress detection, presenting an inverse relationship between signal analysis duration and the effectiveness of stress detection using physiological signals. Conversely, concerning the utilization of EEG signals for stress detection, the performance consistently diminishes as the time period involved in the analysis decreases. On the other hand, Linear Discriminant Analysis (LDA) proved to be the most effective classifier for identifying stress levels within EEG signals, particularly when the time periods involved in the analysis exceeded 4 seconds. Specifically, a 60-second time window using LDA and Naive Bayes algorithms demonstrated flawless accuracy at 100%, whereas a shorter 4-second window achieved an accuracy of 96.3% with KNN. This implies that more extended durations enhance the discriminatory capability for various types of stress within EEG signals. Conversely, when the time periods involved in data analysis are shorter, the Decision Tree classifier excelled in effectively discriminating stress levels among all classifiers.

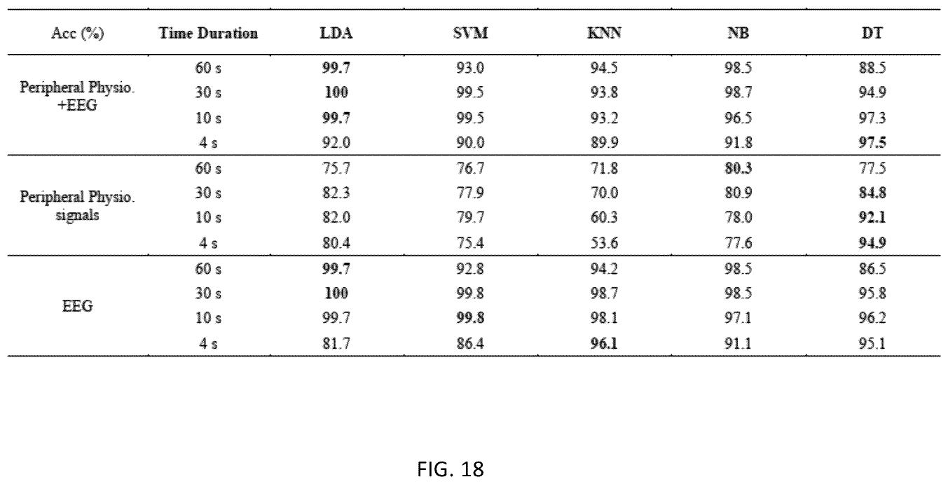

The table shown in FIG. 18 shows the performance of multi-level stress detection using different classifiers. Similar to the stress type detection, when stress detection solely utilizes physiological signals, the Decision Tree classifier emerged as the most effective among all classifiers considered, achieving an impressive average accuracy of 94.9% across the diverse subjects involved in the study. Contrastingly, when focusing on stress detection through EEG signals, the Decision Tree classifier did not exhibit the same robust performance. Instead, Linear Discriminant Analysis (LDA) emerged as the top performer for both 60-second and 30-second signal periods. The accuracy achieved by LDA was notable; however, a significant drop from 100% to 81.7% occurred when the signal analysis duration was reduced from 60 seconds to 4 seconds for stress detection. This divergence underscores the sensitivity of EEG signal-based stress detection when relying on EEG signals, especially on the development of the potential of real-time stress detection. In general, the combined detection utilizing both physiological and EEG features showed apparent improvement compared to relying on individual physiological or EEG features alone. Furthermore, it enhanced the efficiency and accuracy of multi-level stress detection, even within a short analysis period. LDA consistently outperformed all classifiers when using 60-second, 30-second, and 10-second signal durations, while the Decision Tree classifier excelled in effectively discriminating stress when the analysis time dropped to 4 seconds. This dynamic interplay highlights the adaptability and potential of combined features for more robust stress detection methodologies. It is perspective that the integration of physiological and EEG signals offers a comprehensive and promising approach for stress detection across various time scales, with extended periods enhancing classification accuracy.

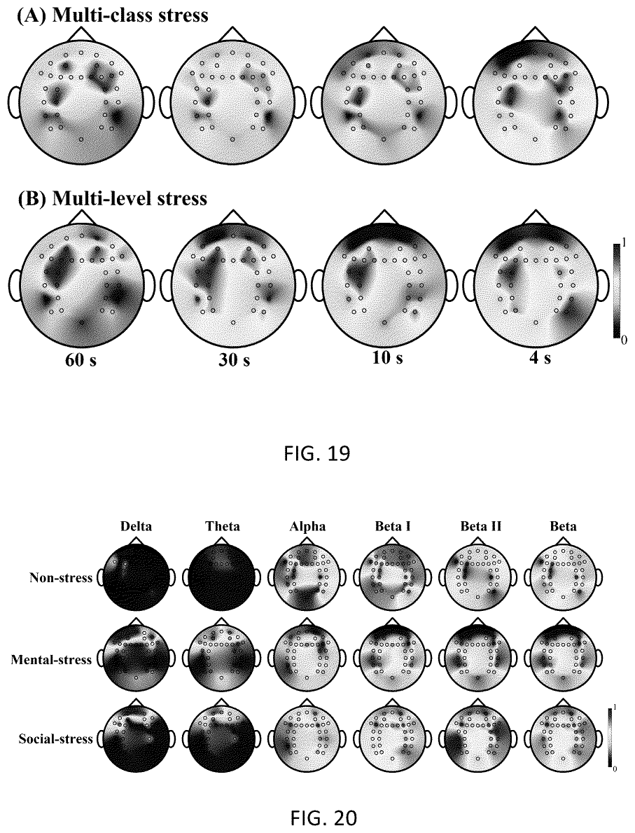

FIG. 19 provides an initial understanding of the spatial distribution of essential features extracted from EEG across the brain. The unique spatial distributions identified from stress types versus stress levels can help emphasize the importance of considering optimal features and approaches in future research endeavors. The determination of the significance of features from each EEG channel plays a crucial role in understanding stress types and levels. This is achieved through the estimation of mutual information between these features and the corresponding stress types/levels. Mutual information is used to assess the dependence between EEG features, and it helps identify the most representative features from EEG signals based on entropy concepts. By quantifying the statistical dependency between features, the topographical concatenation and similarity of joint probability distribution functions of these features can be estimated, providing insights into the connectivity and interdependence of brain regions during stress (as shown in FIG. 19, which illustrates images of EEG feature importance distribution for multi-class stress detection in (A) and multi-level stress detection in (B). The heatmap represents normalized mutual information score, reflecting the significance of each EEG channel contributing to stress detection).

An interesting observation when considering an extended period of signals for stress evaluation is the concentrated presence of the contribution area around the prefrontal cortex. This suggests that the prefrontal cortex plays a key role in processing and responding to stress. However, when shorter time durations are considered, the contribution area expands to include the frontal lobe, parietal lobe, and temporal lobe. This spatial distribution indicates that different regions of the brain are involved in stress processing at different time scales. Considering the findings presented in the tables shown in FIGS. 17 and 18, it becomes evident that with a longer duration of signals, EEG spectral powers could provide a more condensed and reliable capability for the contribution to a more stable evaluation of stress. This is further supported by the concentrated and prominent feature contributions observed within a 60-second timeframe, as opposed to the more dispersed tomography observed within a 4-second timeframe (as illustrated in FIG. 20). Furthermore, it is evident that there is an asymmetry distribution of features observed from the frontal and temporal lobes in both stress type identification and stress level assessment. In the case of multi-level stress discrimination, the left hemisphere exhibits more significant features compared to other regions.

In addition to examining brain cognition, peripheral signals also play a crucial role in identifying stress. These signals provide an initial response to stress before it is even consciously recognized. Multiple peripheral physiological signals contribute to classifying and identifying stress responses, including heart rate, respiration, blood pressure, muscle tension, EMG, GSR, and oxygen level, etc. By analyzing these signals, we can gain valuable insights into the presence and intensity of stress.

There are five peripheral physiological signals were involved in the study, including temperature, oxygen saturation, heart rate, PPG and GSR. Among these signals, PPG and GSR are time series signals that contain numerous temporal features crucial for identifying and understanding stress. Under a stressful situation, a cascade of hormonal changes and physiological responses lead to an elevation in heart rate, as well as a decrease in body temperature and blood oxygen saturation levels. However, the informative features which can be extracted are limited. While these three parameters are influenced by stress, they still exhibit significant variability that can be affected by other factors such as physical activities, environmental temperatures, etc. On the other hand, when combined with PPG and GSR, which are time-series signals involving dynamic changes, more reliable monitoring and detection of stress can be achieved. This can provide a more comprehensive understanding of an individual's stress levels.

This study included the investigation of both different types and levels of stress. One of the objectives was to explore how physiological features contribute to the identification and differentiation of stress across different time durations. Based on the classification results presented in Table 3 and Table 4, it is evident that the decision tree method achieves superior performance compared to other methods when utilizing peripheral physiology features for stress classification. To gain further insights into the contribution of each feature to the classification process, feature importance was assessed for the decision tree algorithm. This was done by measuring the reduction in entropy values, which represents the change in information provided by each feature in identifying the target value. The estimated feature importance is visualized in FIG. 20, which depicts tomography of relative power activation from different EEG rhythms from subject 2, 60s timeframe. The relative power was estimated from the absolute power to total power ratio.

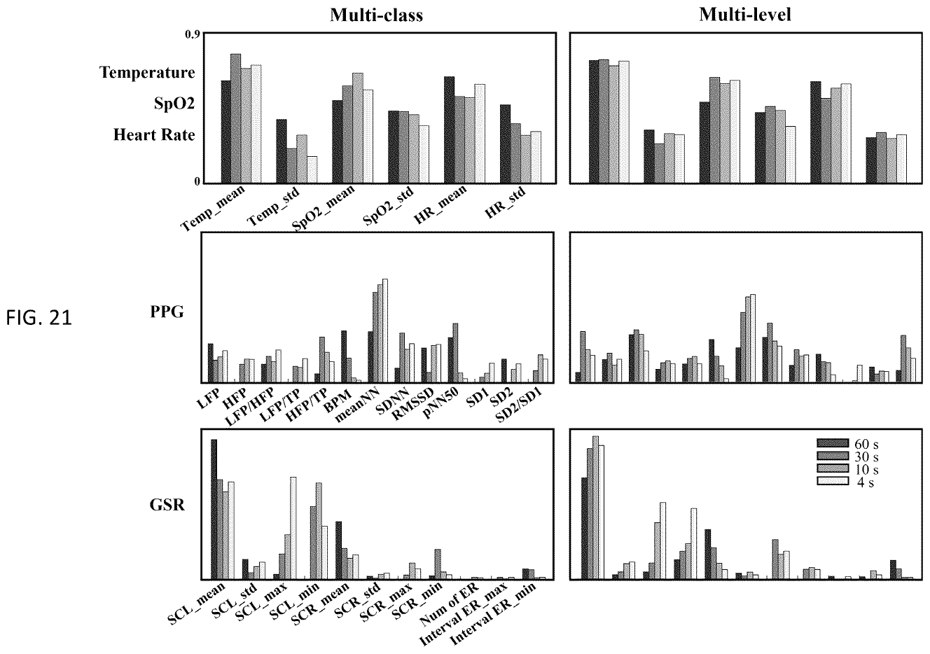

GSR, which represents the electrical properties of skin conductivity, has been demonstrated a high correlation to emotional cognition including stress. The Skin Conductance Level (SCL) derived from GSR directly reflects the general automatic arousal and it has been demonstrated as a reliable indicator of stress. This indicator remains important in stress identification even when the measurement duration is significantly reduced from 60 seconds to 4 seconds, as illustrated in FIG. 21, which shows graphs with feature importance of peripheral physiological features for multi-class and multi-level stress detection across different time durations. The significance of this indicator in stress identification persists, maintaining its effectiveness within very short periods. Notably, SCL emerges as the most influential feature for discriminating stress across varying time lengths derived from GSR. Furthermore, the maximum value of SCL demonstrates enhanced accuracy with decreasing time durations, both in cases of stress type and stress level. This indicates its potential for detecting stress changes within shorter time periods. In general, all SCL features demonstrate an increased contribution to stress discrimination, with this effect particularly pronounced in multi-level stress detection. Conversely, the sensitivity of SCR appears to diminish with decreasing timeframes, especially in terms of the average value of SCR. The effective responses of SCR contribute minimally to stress identification compared to other features. This may be attributed to the fact that SCR responses are particularly pronounced during emotional arousal and are closely linked to stimuli associated with stressors. Literature shows the phasic components of GSR have established strong direct correlations with theta waves (<4 Hz) and high inverse correlation with delta waves (4-8 Hz).

Heart rate is another crucial factor that could indicate stress conditions, lots of studies have demonstrated its efficacy in the detection of stress. Heart rate variability is a reliable primary metric for assessing ANS activity in stress identification, with its fluctuations reflecting an individual's capacity to adapt to changes in the intervals between successive peaks. Decreases in short-term HRV are indicative of acute stress, suggesting a negative impact during stressful situations. Pulse rate variability (PRV), recorded from the PPG sensor, is an alternative method for assessing ANS activity, which reflects changes in arterial blood volume during each cardiac cycle. Previous studies have demonstrated the correlations between PPG features and stress. Some disagreements are also found in previous studies which impose caution when comparing metrics from different sensors and short-term versus chronic stress cases.

In this study, time, frequency, and geometric domain features were extracted and analyzed from PPG signals to evaluate both the levels and types of induced stress. In general, the features extracted from the time domain show greater importance compared to other feature domains (FIG. 21). When considering multi-level classification, a slightly higher importance of the frequency and geometrical features can be observed, with time domain features playing a more significant role in multi-class classification. Furthermore, in the multi-class case, an increased contribution can be observed from meanNN, SDNN, and RMSSD as the time duration decreases from 60 seconds to 4 seconds. This indicates that these features become more influential in capturing stress-related patterns within shorter timeframes. However, in the multi-level case, the opposite trend can be observed, where the contribution of these features decreases as the time duration decreases. Moreover, it is evident that certain features in the time domain are sensitive to the time duration of stress processing. For instance, the average pulse cycle interval (meanNN) becomes more precise with shorter durations both in multi-class and multi-level cases. The frequency domain features demonstrate stability across different time periods, with no significant changes in their contribution observed across various time durations. Conversely, accurate BPM and pNN50 are best captured over longer timeframes, as ultra-short time windows may lead to inaccurate counts due to missed cycles, especially when the heartbeats remain consistent for a prolonged period. In such cases, shorter time durations fail to successfully capture stress-related features.

The functional units described in this specification have been labeled as computing devices. A computing device may be implemented in programmable hardware devices such as processors, digital signal processors, central processing units, field programmable gate arrays, programmable array logic, programmable logic devices, cloud processing systems, or the like. The computing devices may also be implemented in software for execution by various types of processors. An identified device may include executable code and may, for instance, comprise one or more physical or logical blocks of computer instructions, which may, for instance, be organized as an object, procedure, function, or other construct. Nevertheless, the executable of an identified device need not be physically located together but may comprise disparate instructions stored in different locations which, when joined logically together, comprise the computing device and achieve the stated purpose of the computing device. In another example, a computing device may be a server or other computer located within a retail environment and communicatively connected to other computing devices (e.g., POS equipment or computers) for managing accounting, purchase transactions, and other processes within the retail environment. In another example, a computing device may be a mobile computing device such as, for example, but not limited to, a smart phone, a cell phone, a pager, a personal digital assistant (PDA), a mobile computer with a smart phone client, or the like. In another example, a computing device may be any type of wearable computer, such as a computer with a head-mounted display (HMD), or a smart watch or some other wearable smart device. Some of the computer sensing may be part of the fabric of the clothes the user is wearing. A computing device can also include any type of conventional computer, for example, a laptop computer or a tablet computer. A typical mobile computing device is a wireless data access-enabled device (e.g., an iPHONER smart phone, an iPAD device, smart watch, or the like) that is capable of sending and receiving data in a wireless manner using protocols like the Internet Protocol, or IP, and the wireless application protocol, or WAP. This allows users to access information via wireless devices, such as smart watches, smart phones, mobile phones, pagers, two-way radios, communicators, and the like. Wireless data access is supported by many wireless networks, including, but not limited to, Bluetooth, Near Field Communication, CDPD, CDMA, GSM, PDC, PHS, TDMA, FLEX, ReFLEX, iDEN, TETRA, DECT, DataTAC, Mobitex, EDGE and other 2G, 3G, 4G, 5G, and LTE technologies, and it operates with many handheld device operating systems, such as EPOC, Windows CE, FLEXOS, OS/9, JavaOS, iOS and Android. Typically, these devices use graphical displays and can access the Internet (or other communications network) on so-called mini- or micro-browsers, which are web browsers with small file sizes that can accommodate the reduced memory constraints of wireless networks. In a representative embodiment, the mobile device is a cellular telephone or smart phone or smart watch that operates over GPRS (General Packet Radio Services), which is a data technology for GSM networks or operates over Near Field Communication e.g. Bluetooth. In addition to a conventional voice communication, a given mobile device can communicate with another such device via many different types of message transfer techniques, including Bluetooth, Near Field Communication, SMS (short message service), enhanced SMS (EMS), multi-media message (MMS), email WAP, paging, or other known or later-developed wireless data formats. Although many of the examples provided herein are implemented on smart phones, the examples may similarly be implemented on any suitable computing device, such as a computer.

An executable code of a computing device may be a single instruction, or many instructions, and may even be distributed over several different code segments, among different applications, and across several memory devices. Similarly, operational data may be identified and illustrated herein within the computing device, and may be embodied in any suitable form and organized within any suitable type of data structure. The operational data may be collected as a single data set, or may be distributed over different locations including over different storage devices, and may exist, at least partially, as electronic signals on a system or network.

The described features, structures, or characteristics may be combined in any suitable manner in one or more embodiments. In the following description, numerous specific details are provided, to provide a thorough understanding of embodiments of the disclosed subject matter. One skilled in the relevant art will recognize, however, that the disclosed subject matter can be practiced without one or more of the specific details, or with other methods, components, materials, etc. In other instances, well-known structures, materials, or operations are not shown or described in detail to avoid obscuring aspects of the disclosed subject matter.

As used herein, the term “memory” is generally a storage device of a computing device. Examples include, but are not limited to, read-only memory (ROM) and random access memory (RAM).

The device or system for performing one or more operations on a memory of a computing device may be a software, hardware, firmware, or combination of these. The device or the system is further intended to include or otherwise cover all software or computer programs capable of performing the various heretofore-disclosed determinations, calculations, or the like for the disclosed purposes. For example, exemplary embodiments are intended to cover all software or computer programs capable of enabling processors to implement the disclosed processes. Exemplary embodiments are also intended to cover any and all currently known, related art or later developed non-transitory recording or storage mediums (such as a CD-ROM, DVD-ROM, hard drive, RAM, ROM, floppy disc, magnetic tape cassette, etc.) that record or store such software or computer programs. Exemplary embodiments are further intended to cover such software, computer programs, systems and/or processes provided through any other currently known, related art, or later developed medium (such as transitory mediums, carrier waves, etc.), usable for implementing the exemplary operations disclosed below.

In accordance with the exemplary embodiments, the disclosed computer programs can be executed in many exemplary ways, such as an application that is resident in the memory of a device or as a hosted application that is being executed on a server and communicating with the device application or browser via a number of standard protocols, such as TCP/IP, HTTP, XML, SOAP, REST, JSON and other sufficient protocols. The disclosed computer programs can be written in exemplary programming languages that execute from memory on the device or from a hosted server, such as BASIC, COBOL, C, C++, Java, Pascal, or scripting languages such as JavaScript, Python, Ruby, PHP, Perl, or other suitable programming languages.

As referred to herein, a computer network may be any group of computing systems, devices, or equipment that are linked together. Examples include, but are not limited to, local area networks (LANs) and wide area networks (WANs). A network may be categorized based on its design model, topology, or architecture. In an example, a network may be characterized as having a hierarchical internetworking model, which divides the network into three layers: access layer, distribution layer, and core layer. The access layer focuses on connecting client nodes, such as workstations to the network. The distribution layer manages routing, filtering, and quality-of-server (QoS) policies. The core layer can provide high-speed, highly-redundant forwarding services to move packets between distribution layer devices in different regions of the network. The core layer typically includes multiple routers and switches.

The present subject matter may be a system, a method, and/or a computer program product. The computer program product may include a computer readable storage medium (or media) having computer readable program instructions thereon for causing a processor to carry out aspects of the present subject matter.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a RAM, a ROM, an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network, or Near Field Communication. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++, Javascript or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

Aspects of the present subject matter are described herein with reference to flowchart illustrations and/or block diagrams of methods, apparatus (systems), and computer program products according to embodiments of the subject matter. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present subject matter. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the embodiments have been described in connection with the various embodiments of the various figures, it is to be understood that other similar embodiments may be used, or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed is:

1. A system for managing substance use disorder of a person, the system comprising:
a biosensor configured to acquire neurophysiological activity data of a person;
a computing device comprising a substance use disorder manager configured to:
implement an application that interfaces with the person for managing the person's substance use disorder;
receive the acquired neurophysiological activity data;
determine that the person reached a substance abuse trigger point based on the acquired neurophysiological activity data; and
operate a user interface to present an interactive function of the application for distracting the person in response to determining that the person reached the subject abuse trigger point.

2. The system of claim 1, wherein the biosensor comprises one of an electroencephalography (EEG) or an electrodermal activity (EDA).

3. The system of claim 1, wherein the biosensor is configured to output an electrical signal indicative of the neurophysiological activity data and to communicate the electrical signal to the computing device.

4. The system of claim 1, wherein the computing device comprises a smartphone.

5. The system of claim 1, wherein the biosensor is configured to be worn by the person.

6. The system of claim 1, further comprising one or more other biosensors configured to acquire other neurophysiological activity data of the person, and
wherein the substance use disorder manager is configured to determine that the person reached a substance abuse trigger point based on the other acquired neurophysiological activity data.

7. The system of claim 1, wherein the user interface comprises one of a display or a speaker.

8. The system of claim 1, wherein the substance use disorder manager is configured to implement an algorithm for determining the substance abuse trigger point based on the acquired neurophysiological activity data.

9. The system of claim 1, wherein the substance use disorder manager is configured to implement an artificial intelligence or machine learning algorithm for determining the substance abuse trigger point based on the acquired neurophysiological activity data.

10. The system of claim 1, wherein the substance use disorder manager is configured to:
idle the interactive function of the application;
enable the interactive function of the application; and
manage whether the interactive function of the application is idled or enabled based on the neurophysiological activity data received from the biosensor.

11. The system of claim 1, wherein the interactive function of the application comprises a stimulus for distracting from substance abuse.

12. The system of claim 1, wherein the interactive function of the application comprises a function that presents a cognitive-behavioral therapy (CBT) interface, a neuro feedback interface, a game, or a neuro stimulation activity to the user.

13. A method for managing substance use disorder of a person, the method comprising:
providing a biosensor configured to acquire neurophysiological activity data of a person;
implementing an application that interfaces with the person for managing the person's substance use disorder;
receiving the acquired neurophysiological activity data;
determining that the person reached a substance abuse trigger point based on the acquired neurophysiological activity data; and
operating a user interface to present an interactive function of the application for distracting the person in response to determining that the person reached the subject abuse trigger point.

14. The method of claim 13, wherein the biosensor comprises one of an electroencephalography (EEG) or an electrodermal activity (EDA).

15. The method of claim 13, wherein the biosensor is configured to output an electrical signal indicative of the neurophysiological activity data and to communicate the electrical signal to the computing device.

16. The method of claim 13, wherein the biosensor is configured to be worn by the person.

17. The method of claim 13, further comprising:
providing one or more other biosensors configured to acquire other neurophysiological activity data of the person; and
determining that the person reached a substance abuse trigger point based on the other acquired neurophysiological activity data.

18. The method of claim 13, wherein the user interface comprises one of a display or a speaker.

19. The method of claim 13, further comprising implementing an algorithm for determining the substance abuse trigger point based on the acquired neurophysiological activity data.

20. The method of claim 13, further comprising implementing an artificial intelligence or machine learning algorithm for determining the substance abuse trigger point based on the acquired neurophysiological activity data.

21. The method of claim 13, further comprising:
idling the interactive function of the application;
enabling the interactive function of the application; and
managing whether the interactive function of the application is idled or enabled based on the neurophysiological activity data received from the biosensor.

22. The method of claim 13, wherein the interactive function of the application comprises a stimulus for distracting from substance abuse.

23. The method of claim 13, wherein the interactive function of the application comprises a function that presents a cognitive-behavioral therapy (CBT) interface, a neuro feedback interface, a game, or a neuro stimulation activity to the user.
